(19) United States
(12) Patent Application Publication
(10) Pub. No.: US 2025/0195482 A1
(43) Pub. Date: Jun. 19, 2025

(54) METHOD AND COMPOSITION FOR IMPROVING HEALTH OF HAIR FOLLICLES, SCALP OR HAIR OF MAMMALS

(71) Applicant: NANJING NUTRABUILDING BIO-TECH CO., LTD., Nanjing (CN)

(72) Inventors: Mingru WANG, Nanjing (CN); Kylin LIAO, Nanjing (CN)

(21) Appl. No.: 19/064,201

(22) Filed: Feb. 26, 2025

Related U.S. Application Data
(63) Continuation of application No. PCT/CN2023/102428, filed on Jun. 26, 2023.

(30) Foreign Application Priority Data
Aug. 30, 2022 (CN) 202211048670.1

(51) Int. Cl. A61K 31/4375; A61K 8/49; A61P 17/00; A61P 31/04; A61Q 5/00; A61Q 7/00; A61Q 17/00

(52) U.S. Cl. CPC A61K 31/4375; A61K 8/49; A61P 17/00; A61P 31/04; A61Q 5/006; A61Q 5/008; A61Q 7/00; A61Q 17/005

(57) ABSTRACT

The present invention discloses a method and composition for improving hair follicle, scalp or hair health in a mammal and the related application thereof in preparing personal care product, cosmetic or medicine for improving hair follicle, scalp or hair health of a mammal. The method and composition of the invention inhibits the growth of a microorganism related to hair follicle, scalp or hair health, and/or eliminates inflammation, and/or inhibits folliculitis, scalp itching, dandruff, scalp oiliness, rashes, skin itching, scalp flaking, hair loss, tinea capitis, hypotrichosis, oligotrichosis, and lichen planopilaris.

### CROSS REFERENCE TO RELATED APPLICATION

This application is a continuation application of International Patent Application No. PCT/CN2023/102428, filed on Jun. 26, 2023, which claims the priority of the Chines patent application No. CN2022110486701, filed on Aug. 30, 2022, the contents of all of which are incorporated herein by reference in their entirety.

### FIELD OF THE INVENTION

The present invention belongs to the technical field of cosmetics and personal care products, and specifically relates to a method and composition for improving hair follicle, scalp or hair health in a mammal and the related application thereof in preparing personal care product, cosmetic or medicine for improving hair follicle, scalp or hair health of a mammal.

### BACKGROUND OF THE INVENTION

The billions and trillions of microbes combine to form a “microbiome,” which are found throughout our bodies-especially in our guts, stomachs, and skin. The health of microbiomes determines how healthy we are as human beings. The scalp microbiome is a simple community of organisms that grow on the skin on top of the head. A wide variety of bacteria work together, each making its own contribution to the health of scalp. If this delicate balance is thrown off, the scalp's microbiota is not normal, then there will be rashes, flaking of the scalp, itchy skin, androgenetic alopecia, and so on.

Microbiology research study indicates that androgenetic alopecia is linked to the bacteria found on hair follicles. The research subjects with androgenetic alopecia areata have the same species of yeast (Malassezia) as those with dandruff. In addition, commensal bacterial species such as Malassezia, Propionibacterium acnes, Staphylococcus aureus and Staphylococcus epidermidis are the main bacteria associated with scalp health. Reducing or inhibiting the growth of these microbiome will significantly improve scalp health and prevent hair loss.

Most scalp health problems will develop into inflammation, such as folliculitis or seborrheic dermatitis. Dihydroberberine (DHB) is an excellent anti-inflammatory and antibacterial substance. In addition, it has been reported that DHB has a good regulatory effect on gut microbiota. However, there are no relevant studies on the interaction between DHB and scalp microbiota.

It would be desirable to provide improved methods and compositions for improving hair follicle, scalp or hair health by modulating certain scalp microbiota, as well as actively treating or inhibiting scalp inflammation.

### SUMMARY OF THE INVENTION

In order to achieve the above object, in one aspect, the present invention provides a method for improving hair follicle, scalp or hair health in a mammal comprising administering a composition to the mammal, the composition comprises an effective amount of dihydroberberine or a pharmaceutically acceptable salt, acid, ester, analog or derivative thereof. In some embodiments, the composition further comprises a pharmaceutically acceptable carrier.

In some embodiments, the composition inhibits the growth of a microorganism related to hair follicle, scalp or hair health and/or eliminates inflammation.

In some embodiments, the microorganism may be a lipophilic fungus or a Gram-positive bacterium, preferably a lipophilic fungus or a Gram-positive bacterium related to hair follicle, scalp or hair health; the inflammation can be hair follicle, scalp or hair health-related inflammation. In preferred embodiments, the microorganism is selected from one or more of the following: M. globosa, M. restricta, Propionibacterium acnes, Staphylococcus aureus and Staphylococcus epidermidis; the inflammation is folliculitis or seborrheic dermatitis.

In some embodiments, the composition inhibits folliculitis, scalp itching, dandruff, scalp oiliness, rashes, skin itching, scalp flaking, hair loss, tinea capitis, hypotrichosis, oligotrichosis, and lichen planopilaris.

In some embodiments, the composition is prepared into personal care products, cosmetics, or medicines.

In some embodiments, the composition is in the form of lotion, cream, ointment, essence, paste, gel, solution, dispersion, spray, suspension, emulsion, foam, patch, powder, film, elixir, tincture and liniment.

In some embodiments, the dihydroberberine or a pharmaceutically acceptable salt, acid, ester, analog or derivative thereof is formulated to be administered in an amount of 50-5000 mg, preferably 100-2500 mg, more preferably 200-1300 mg, most preferably 200-800 mg per day.

In some embodiments, the composition is administered topically, transdermally or subcutaneously. In some embodiments, the composition is administered orally and externally. For oral administration, the composition of the present invention can be in any suitable form, including solution, tablet, gel capsule, capsule or alternative nutritious food or nutritional supplement. For external administration, the composition will be applied to the skin part to be treated, especially the face, neck or hand, once or several times a day. The forms of the composition include skin lotion, skin softener, skin toner, skin cream, sunscreen, essence, mask pack, mask sheet, soap, shampoo, cleaning foam, cleaning lotion, cleaning cream, ointment, gel, massage cream and etc.

In another aspect, the present invention provides a composition comprising dihydroberberine or a pharmaceutically acceptable salt, acid, ester, analog or derivative thereof, wherein the composition is used for improving hair follicle, scalp or hair health in a mammal. In some embodiments, the composition further comprises a pharmaceutically acceptable carrier.

In some embodiments, the composition inhibits the growth of a microorganism related to hair follicle, scalp or hair health and/or eliminates inflammation.

In some embodiments, the microorganism may be a lipophilic fungus or a Gram-positive bacterium, preferably a lipophilic fungus or a Gram-positive bacterium related to hair follicle, scalp or hair health; the inflammation can be hair follicle, scalp or hair health-related inflammation. In preferred embodiments, the microorganism is selected from one or more of the following: M. globosa, M. restricta, Propionibacterium acnes, Staphylococcus aureus and Staphylococcus epidermidis; the inflammation is folliculitis or seborrheic dermatitis.

In some embodiments, the composition inhibits folliculitis, scalp itching, dandruff, scalp oiliness, rashes, skin itching, scalp flaking, hair loss, tinea capitis, hypotrichosis, oligotrichosis, and lichen planopilaris.

In some embodiments, the dihydroberberine or a pharmaceutically acceptable salt, acid, ester, analog or derivative thereof is formulated to be administered in an amount of 50-5000 mg, preferably 100-2500 mg, more preferably 200-1300 mg, most preferably 200-800 mg per day. In some embodiments, the composition is administered by a route such as topical, transdermal or subcutaneous administration. In some embodiments, the composition is prepared into personal care products, cosmetics, or medicines. In some embodiments, the composition is in the form of lotion, cream, ointment, essence, paste, gel, solution, dispersion, spray, suspension, emulsion, foam, patch, powder, film, elixir, tincture and liniment.

In another aspect, the present invention provides use of a composition in preparing personal care products, cosmetic or medicine for improving hair follicle, scalp or hair health in a mammal, wherein the composition comprises an effective amount of dihydroberberine or a pharmaceutically acceptable salt, acid, ester, analog or derivative thereof. In some embodiments, the composition further comprises a pharmaceutically acceptable carrier. In some embodiments, the composition inhibits the growth of a microorganism related to hair follicle, scalp or hair health and/or eliminates inflammation. In some embodiments, the microorganism may be a lipophilic fungus or a Gram-positive bacterium, preferably a lipophilic fungus or a Gram-positive bacterium related to hair follicle, scalp or hair health; the inflammation can be hair follicle, scalp or hair health-related inflammation. Preferably, the microorganism is selected from one or more of the following: M. globosa, M. restricta, Propionibacterium acnes, Staphylococcus aureus and Staphylococcus epidermidis; the inflammation is folliculitis or seborrheic dermatitis.

In some embodiments, the composition is administered to the mammal in an amount of 50-5000 mg, preferably 100-2500 mg, more preferably 200-1300 mg, and most preferably 200-800 mg of dihydroberberine or a pharmaceutically acceptable salt, acid, ester, analog or derivative thereof per day. In some embodiments, the composition is administered by a route such as topical, transdermal or subcutaneous administration. In some embodiments, the composition inhibits folliculitis, scalp itching, dandruff, scalp oiliness, rashes, skin itching, scalp flaking, hair loss, tinea capitis, hypotrichosis, oligotrichosis, and lichen planopilaris. In some embodiments, the composition is in the form of lotion, cream, ointment, essence, paste, gel, solution, dispersion, spray, suspension, emulsion, foam, patch, powder, film, elixir, tincture and liniment.

This summary is provided to introduce the selection of concepts in a simplified form, which are further described in the detailed description below. Summary is not intended to identify key features or essential features of the claimed subject matter, nor is it intended to be used to limit the scope of the claimed subject matter.

### BRIEF DESCRIPTION OF DRAWINGS

FIG. 1 is an optical density diagram of M. globosa at 600 nm after being treated with DHB of various concentrations.

FIG. 2 is an optical density diagram of M. restricta at 600 nm after being treated with DHB of various concentrations.

FIG. 3 is an optical density diagram of Propionibacterium acnes at 530 nm after being treated with DHB of various concentrations.

FIG. 4 is an optical density diagram of Staphylococcus aureus at 530 nm after being treated with DHB of various concentrations.

FIG. 5 is an optical density diagram of Staphylococcus epidermidis at 530 nm after being treated with DHB of various concentrations.

### DETAILED DESCRIPTION OF THE INVENTION

Reference will now be made in detail to the preferred embodiments of the invention, examples of which are further illustrated. While the invention will be described in conjunction with the preferred embodiments, it will be understood that they are not intended to limit the invention to these embodiments. To the contrary, the invention is intended to cover alternatives, modifications and equivalents, which may be included within the spirit and scope of the invention as defined by the claims. Furthermore, in the detailed description of the present invention, numerous specific details are set forth in order to provide a thorough understanding of the present invention. However, it will be obvious to one of ordinary skill in the art that the present invention may be practiced without these specific details. In other instances, well known methods, procedures, components, and other features have not been described in detail as not to unnecessarily obscure aspects of the present invention.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the term “comprise” or “include” or their conjugations, refer to a situation where said terms are used in their non-limiting sense to mean that items following the word are included, but items not specifically mentioned are not excluded. It also encompasses the more limiting verb ‘to consist essentially of’ and ‘to consist of’.

As used herein, the term “mammal” or “subject” may be used interchangeably to refer to any animal to which the presently disclosed methods and compositions may be applied or administered. The animal may have an illness or other disease, but the animal does not need to be sick to benefit from the presently disclosed methods and compositions. As such, any animal may apply the disclosed compositions, or be a recipient of the disclosed methods. Although the animal subject is preferably a human, the methods and compositions of the invention have application in veterinary medicine as well, e.g., for the treatment of domesticated species such as canine, feline, murine, and various other pets; farm animal species such as bovine, equine, ovine, caprine, porcine, etc.; and wild animals, e.g., in the wild or in a zoological garden, such as non-human primates.

As used herein, the term “administration” refers to the process of delivering a disclosed composition or active ingredient to a subject. The composition of the present invention is preferably administered by topical, transdermal or subcutaneous administration, but it can also be administered by other conventional routes to exert the desired effect.

As used herein, the term “effective amount” refers to an amount that is required to achieve the effect as taught herein. An effective amount herein includes, but is not limited to the amount necessary to improve hair follicle, scalp or hair health in a mammal; and/or the amount necessary to inhibit the growth of a microorganism related to hair follicle, scalp or hair health and/or eliminate inflammation; and/or the amount necessary to inhibit folliculitis, scalp itching, dandruff, scalp oiliness, rashes, skin itching, scalp flaking, hair loss, tinea capitis, hypotrichosis, oligotrichosis, and lichen planopilaris. In accordance with the present disclosure, a suitable single dose size is that which, when administered one or more times over a suitable period of time, achieves the above-described effects.

As used herein, the term “pharmaceutically acceptable” means pharmaceutically, physiologically, cosmetically, or cosmetologically acceptable, and refers to those compositions or combinations of agents, materials, or compositions, and/or their dosage forms, which are within the scope of sound medical judgment, suitable for use in contact with the tissues of human beings and animals, compatible with other components of the composition, without excessive toxicity, irritation, allergic response, or other problem or complication, commensurate with a reasonable benefit/risk ratio.

As used herein, the term “carrier” may include liquid or solid fillers, diluents, excipients, solvents, encapsulating materials, buffers, stabilizers, preservatives, oils, surfactants, gelling agents, antioxidants, chelating agents, viscosity enhancers, ultraviolet absorbers, pH adjusters, pigments, fragrances, and the like. The carrier used in the present invention is preferably one that is suitable for use in personal care products, cosmetics, beauty products, or medicines.

The method of the present invention comprises administering at least 50 mg, typically 50-5000 mg, preferably 100-2500 mg, more preferably 200-1300 mg, and most preferably 200-800 mg of DHB or a pharmaceutically acceptable salt, acid, ester or derivative thereof every day, depending on the specific preparation and form. The amount to be administered can also vary according to factors such as sensitivity, age, sex and weight of the subject, specific reaction, etc. One or more doses can be administered once or more times a day or at a suitable frequency for any period of time. For example, an effective dose can be administered daily for one day, several days, many days or indefinitely.

The following examples are illustrative of selected embodiments of the present invention and are not meant to limit the scope of the invention.

### EXAMPLES

Growth inhibition of M. globosa, M. restricta, Propionibacterium acnes, Staphylococcus aureus and Staphylococcus epidermidis was tested using 50-ml tubes with 10 ml of mDixon medium. Cultures were inoculated to an optical density (OD) of 0.2, the above microorganisms were loaded into each culture dish, and challenged with test material (control and DHB with different concentrations: 0.001 μM, 0.01 μM, 0.1 μM, 1 μm, 10 μM, 100 μM and 1000 μM). After 48 h of shaking at 30° C., the OD was measured by spectrophotometer and recorded.

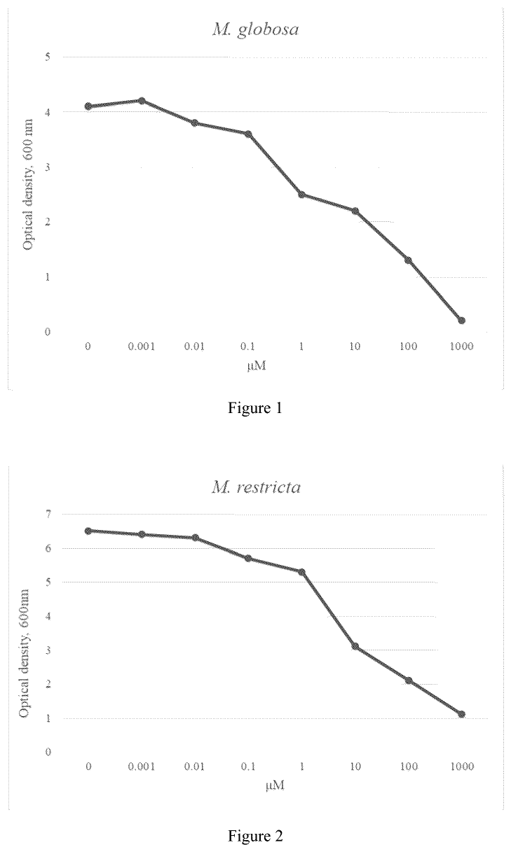

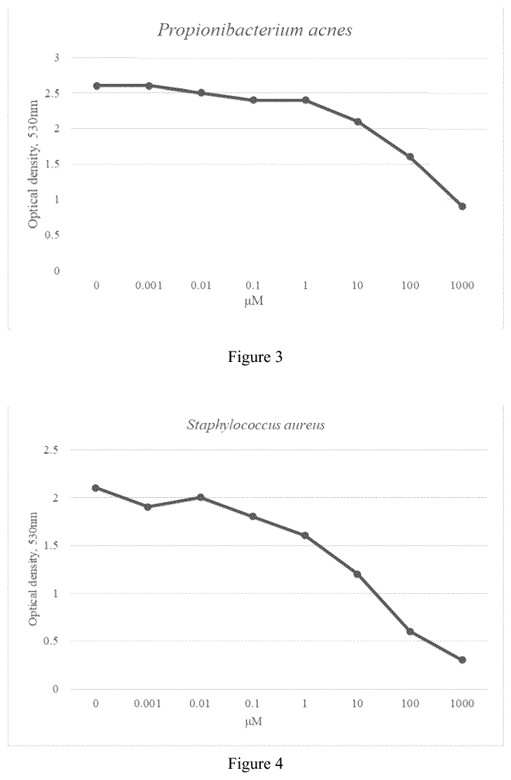

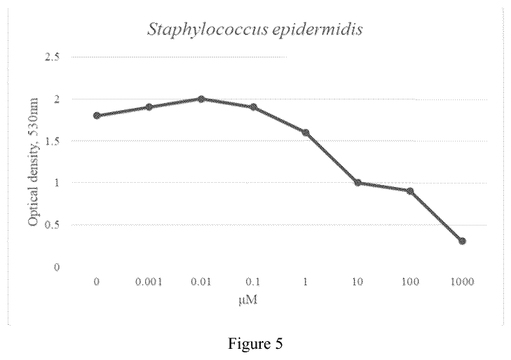

As shown in FIGS. 1-2, FIGS. 1 and 2 are optical density diagrams of M. globosa and M. restricta at 600 nm after being treated with DHB of various concentrations, respectively. After DHB treatment, the optical density of M. globosa and M. restricta dishes decreased. The higher the concentration of DHB, the lower the optical density. Under the treatment of 1000 μM DHB, the optical density of M. globosa even decreased from 4.1 in the control treatment to 0.2, while that of M. restricta also decreased from 6.5 in the control treatment to 1.1, indicating that DHB can significantly inhibit the growth of lipophilic fungi such as M. globosa and M. restricta. As shown in FIGS. 3-5, FIGS. 3, 4 and 5 are optical density diagrams of Propionibacterium acnes, Staphylococcus aureus and Staphylococcus epidermidis at 530 nm after being treated with DHB of various concentrations, respectively. After DHB treatment, the optical density of Propionibacterium acnes, Staphylococcus aureus and Staphylococcus epidermidis dishes decreased. The higher the concentration of DHB, the lower the optical density. Under the treatment of 1000 μM DHB, the optical density of Propionibacterium acnes decreased from 2.6 in the control treatment to 0.9, while the optical density of Staphylococcus aureus and Staphylococcus epidermidis even decreased from 2.1 and 1.8 respectively in the control treatment to 0.3, indicating that DHB can significantly inhibit the growth of Gram-positive bacteria such as Propionibacterium acnes, Staphylococcus aureus and Staphylococcus epidermidis.

The experiment confirmed that DHB has a positive effect on the regulation of scalp microbiota, and significantly reduces the levels of Malassezia, Propionibacterium acnes, Staphylococcus aureus and Staphylococcus epidermidis; relieves or eliminates scalp inflammation such as folliculitis, seborrheic dermatitis, etc.; and then repairs scalp or subcutaneous barrier damage; and also has positive treatment or inhibition effects on scalp itching, dandruff, scalp oiliness, rashes, skin itching, scalp flaking, hair loss, tinea capitis, hypotrichosis, oligotrichosis, lichen planopilaris, etc.; and has positive regulation effects on hair follicle, scalp or hair health.

In the present invention, after dihydroberberine treatment, other scalp microbiota can also be inhibited, including other lipophilic fungi or Gram-positive bacteria related to hair follicle, scalp or hair health.

Although specific embodiments and examples of this invention have been illustrated herein, it will be appreciated by those skilled in the art that any modifications and variations can be made without departing from the spirit of the invention. The examples and illustrations above are not intended to limit the scope of this invention. Any combination of embodiments of this invention, along with any obvious their extension or analogs, are within the scope of this invention. Further, it is intended that this invention encompass any arrangement, which is calculated to achieve that same purpose, and all such variations and modifications fall within the scope of the appended claims.

What is claimed is:

1. A method for improving hair follicle, scalp or hair health in a mammal comprising administering a composition to the mammal, the composition comprises an effective amount of dihydroberberine or a pharmaceutically acceptable salt, acid, ester, analog or derivative thereof.

2. The method of claim 1, wherein the composition inhibits growth of a microorganism related to hair follicle, scalp or hair health and/or eliminates inflammation.

3. The method of claim 2, wherein the microorganism is a lipophilic fungus or a Gram-positive bacterium.

4. The method of claim 2, wherein the microorganism is selected from one or more of the following: M. globosa, M. restricta, Propionibacterium acnes, Staphylococcus aureus and Staphylococcus epidermidis; and the inflammation is folliculitis or seborrheic dermatitis.

5. The method of claim 1, wherein the composition inhibits folliculitis, scalp itching, dandruff, scalp oiliness, rashes, skin itching, scalp flaking, hair loss, tinea capitis, hypotrichosis, oligotrichosis, and lichen planopilaris.

6. The method of claim 1, wherein the composition is prepared into personal care products, cosmetics, or medicines.

7. The method of claim 1, wherein the composition is in a form of lotion, cream, ointment, essence, paste, gel, solution, dispersion, spray, suspension, emulsion, foam, patch, powder, film, elixir, tincture and liniment.

8. The method of claim 1, wherein the dihydroberberine or the pharmaceutically acceptable salt, acid, ester, analog or derivative thereof is formulated to be administered in an amount of 50-5000 mg per day.

9. The method of claim 1, wherein the composition is administered topically, transdermally or subcutaneously.

10. The method of claim 1, wherein the composition further comprises a pharmaceutically acceptable carrier.
